(19) United States
(12) Patent Application Publication
(10) Pub. No.: US 2024/0009348 A1
(43) Pub. Date: Jan. 11, 2024

(54) MULTI-LAYER AMNION PRODUCT, RELATED DEVICES, AND RELATED METHODS

(71) Applicant: Axogen Corporation, Alachua, FL (US)

(72) Inventors: Kiri K. HAMAKER, Tampa, FL (US); Alec SIMON, Alachua, FL (US); Curt DEISTER, Alachua, FL (US); Erick DEVINNEY, Alachua, FL (US); Mark FRIEDMAN, Alachua, FL (US)

(73) Assignee: Axogen Corporation, Alachua, FL (US)

(21) Appl. No.: 18/347,279

(22) Filed: Jul. 5, 2023

Related U.S. Application Data
(60) Provisional application No. 63/367,940, filed on Jul. 8, 2022.

(51) Int. Cl. A61L 27/18; A61L 27/56; A61L 27/36

(52) U.S. Cl. CPC A61L 27/18; A61L 27/56; A61L 27/3604; A61L 2430/32; A61L 2430/34

(57) ABSTRACT

A multi-layer amnion product may include a plurality of layers oriented on top of one another, wherein the plurality of layers includes at least one chorion membrane layer and at least two amnion layers. The plurality of layers may include at least four layers and less than ten layers. At least two of the at least two amnion layers may form outermost layers of the plurality of layers. Each amnion layer may include an epithelial layer, a basement membrane, and a fibroblast layer, and each chorion layer may include a reticular layer and a basement membrane.

### CROSS-REFERENCE TO RELATED APPLICATIONS

This patent application claims the benefit under 35 U.S.C. § 120 to U.S. Provisional Patent Application No. 63/367,940, filed on Jul. 8, 2022, the entirety of which is incorporated herein by reference.

### TECHNICAL FIELD

Various embodiments of the present disclosure relate generally to tissue sheets, covers, wraps, connectors, grafts, or protectors (collectively “tissue products”), and, more particularly, to multi-layer tissue products, e.g., amnion tissue products formed of amnion tissue or amnion and chorion tissue, as well as related methods of manufacturing and methods of use.

### BACKGROUND

Following one or more of tissue injury, tissue repair, and/or tissue reconstruction, protecting a damaged tissue area may facilitate the healing process. For example, in the case of nerve tissue, failure to cover and/or isolate a nerve repair or nerve injury site may lead to undesired axonal growth into surrounding areas, which may result in soft tissue attachment and scarring. By protecting a nerve repair or injury site (e.g., through covering and isolation), undesired axonal growth may be inhibited (e.g., reduced or eliminated), and, in some instances, healing time may be decreased by directing axonal growth towards a preferred nerve regeneration site, instead of non-targeted areas. Further, such techniques can also help to provide reinforcement to a nerve repair or injury site and help to inhibit separation of coapted nerves. In order to provide protection and covering at a nerve repair or injury site, membranous tissue grafts in the form of tubes, conduits, sheets for wrapping (i.e., wraps), or other forms for supporting and reinforcing microsurgical repairs of injured nerves may be used. As discussed in detail below, a multi-layer amnion product may be used to help repair tissue, for example, to help repair nerves.

### SUMMARY

The present invention is directed to multi-layer tissue products and methods of making such products. The multi-layer tissue product may be any type of tissue, for example amnion or amnion and chorion tissue. According to aspects of this disclosure, a multi-layer amnion product may be formed and/or used to help repair tissue (e.g., nerves).

In one aspect, a dehydrated multi-layer amnion product may comprise a plurality of layers oriented on top of one another, wherein the plurality of layers includes at least four layers and less than ten layers made up of at least one chorion membrane layer and at least two amnion layers, and wherein at least two of the at least two amnion layers form outermost layers of the plurality of layers. Each amnion layer may comprise an epithelial layer, a basement membrane, and a fibroblast layer, and each chorion layer may comprise a reticular layer and a basement membrane.

In various aspects, the plurality of layers may comprise five, six, or seven layers; the multi-layer amnion product may comprise at least three amnion layers, wherein at least two of the at least three amnion layers are adjacent one another, and the adjacent amnion layers are oriented so that the epithelial layer of a first adjacent amnion layer is oriented facing the fibroblast layer of a second adjacent amnion layer; the multi-layer amnion product may comprise at least three amnion layers, wherein at least two of the at least three amnion layers are adjacent one another, and the adjacent amnion layers are oriented so that the fibroblast layer of a first adjacent amnion layer is oriented facing the fibroblast layer of a second adjacent amnion layer; each of the outermost amnion layers of the plurality of layers may be oriented such that the epithelial layer is facing outward; and the at least two amnion layers and the at least one chorion layer may be obtained from a human or a non-human source.

In some aspects, a method of making a multi-layer amnion product may comprise washing amnion and chorion layers, wherein the amnion and chorion layers are obtained from an amniotic sac and a placenta, cutting the amnion and chorion layers, and overlaying the washed and cut amnion and chorion layers on top of one another to form a multi-layer sheet having at least four layers and less than ten layers, wherein the washed and cut amnion and chorion layers are oriented so that outermost layers of the multi-layer sheet are amnion layers. The method may also comprise drying the multi-layer sheet to form a dehydrated sheet, and sterilizing the multi-layer sheet to form the multi-layer amnion product.

Various aspects of the method may include wherein washing the amnion and chorion layers includes using two cleaning solutions; a first of the two cleaning solutions may include about 0.5% Tween 20 (v/v), about 0.05% polyhexamethylene biguanide (w/v) or alternately about 0.5% (w/v) chlorhexidine, about 0.9% NaCL (w/v), about 10 mM Bis-Tris Propane, and a solution of about pH 6.7+/−0.1; a first of the two cleaning solutions may include about 0.5% Tween 20 (v/v) and about 10.0% NaCl (w/v) and have a pH of about 4.5-7.0; a second of the two cleaning solutions may include about 0.144 g/L KH₂PO₄, about 9 g/L NaCl, and about 0.795 g/L Na₂HPO₄; a second of the two cleaning solutions may include about 0.9% NaCl (w/v) and have a pH of about 4.5-7.0; at least two adjacent amnion layers may be oriented adjacent one another such that a fibroblast layer of a first adjacent amnion layer is oriented facing a fibroblast layer of a second adjacent amnion layer, or the fibroblast layer of the first adjacent amnion layer is oriented facing an epithelial layer of a second adjacent amnion layer; the multi-layer sheet may comprise five, six, or seven layers; the outermost amnion layers may be oriented such that an epithelial layer of each of the outermost amnion layers is facing outward; harvesting the amnion and chorion layers from the amniotic sac and placenta may include separating the amnion layer at a sponge layer, separating the chorion layer from a decidua of the amniotic sac at a trophoblast layer, and substantially removing trophoblast cells remaining on the chorion layer; cutting the amnion and chorion layers may occur before washing the amnion and chorion layers; the multi-layer sheet may comprise five layers in the orientation of AACCA, ACACA, AACAA, AAACA, or ACCCA, wherein each “A” represents an amnion layer and each “C” represents a chorion layer; the multi-layer sheet may comprise six layers in the orientation of AACCAA, AAACCA, AAAACA, AACAAA, ACCCAA, or ACCCCA, wherein each “A” represents an amnion layer and each “C” represents a chorion layer; the multi-layer sheet may comprise seven layers in the orientation of AAACAAA, AAAACAA, AAAAACA, AACCAAA, AACCCAA, AAACCCA, AACCCCA, or ACCCCCA, wherein each “A” represents an amnion layer and each “C” represents a chorion layer; sterilizing the multi-layer sheet comprises using e-beam radiation to achieve sterility assurance of about 10⁻⁶; drying the multi-layer sheet to form a dehydrated sheet comprises using a vacuum pressure of about 50 mbar to about 350 mbar at a temperature of about 22 degrees Celsius to about 35 degrees Celsius; or the method may further comprise rolling the multi-layer sheet to form a tube.

Various aspects of the disclosure may be drawn to a method of making a multi-layer amnion product comprising washing a plurality of amnion layers, wherein the amnion layers are obtained from an amniotic sac; cutting the amnion layers, and overlaying the washed and cut amnion layers on top of one another to form a multi-layer sheet having at least four layers and less than ten layers, wherein the washed and cut amnion layers are oriented so that outermost layers of the multi-layer sheet are amnion layers, and the outermost amnion layers are oriented such that an epithelial layer of each of the outermost amnion layers is facing outward. The method may further comprise drying the multi-layer sheet to form a dehydrated sheet, and sterilizing the multi-layer sheet to form the multi-layer amnion product.

In various aspects, the multi-layer sheet may comprise five, six, or seven layers; adjacent amnion layers may be oriented so that an epithelial layer of a first adjacent amnion layer is oriented facing a fibroblast layer of a second adjacent amnion layer; or adjacent amnion layers may be oriented so that a fibroblast layer of a first adjacent amnion layer is oriented facing a fibroblast layer of a second adjacent amnion layer.

Some aspects of the disclosure may be drawn to a dehydrated multi-layer amnion product comprising a plurality of layers oriented on top of one another, wherein the plurality of layers includes at least four amnion layers, wherein each amnion layer comprises an epithelial layer, a basement membrane, and a fibroblast layer, and wherein outermost layers of the plurality of layers are amnion layers, and wherein the outermost amnion layers are oriented such that the epithelial layer is facing outward.

In various aspects, the plurality of layers may consist of five amnion layers; the plurality of layers may comprise four, five, six, or seven layers; adjacent amnion layers may be oriented so that the epithelial layer of a first adjacent amnion layer is oriented facing the fibroblast layer of a second adjacent amnion layer; adjacent amnion layers may be oriented so that the fibroblast layer of a first adjacent amnion layer is oriented facing the fibroblast layer of a second adjacent amnion layer; or the amnion layers may be obtained from a human or non-human source.

Additional objects and advantages of the disclosed embodiments will be set forth in part in the description that follows, and in part will be apparent from the description, or may be learned by practice of the disclosed embodiments. The objects and advantages of the disclosed embodiments will be realized and attained by means of the elements and combinations particularly pointed out in the appended claims.

It is to be understood that both the foregoing general description and the following detailed description are exemplary and explanatory only and are not restrictive of the disclosed embodiments, as claimed.

### BRIEF DESCRIPTION OF THE DRAWINGS

The accompanying drawings, which are incorporated in and constitute a part of this specification, illustrate various exemplary embodiments and together with the description, serve to explain the principles of the disclosed embodiments. Drawings included herein may not be drawn to scale.

FIG. 1 is a cross-sectional view of human amniotic placental sac.

FIG. 2 is a perspective view of a multi-layer amnion product, according to one or more embodiments.

FIG. 3A shows a human placental amniotic sac, and FIGS. 3B-3E show hematoxylin and eosin stain histological images of various portions or layers of a harvested amniotic sac.

FIG. 4 is a graph of various properties of different multi-layer amnion products with different numbers of layers.

FIG. 5 is a flow chart of an exemplary method of forming a multi-layer amnion product.

FIG. 6 is a flow chart of another exemplary method of forming a multi-layer amnion product.

### DETAILED DESCRIPTION OF EMBODIMENTS

The present invention provides multi-layer tissue products and methods of making such products. The multi-layer tissue products may be any type of tissue, for example membranous tissue grafts for tissue repair, reconstruction, or protection. Various embodiments of the present disclosure relate generally to a multi-layer amnion products, which may be used to help repair tissue, for example, to help repair nerves.

In this disclosure, the singular forms “a,” “an,” and “the” include plural referents unless the context dictates otherwise. The term “exemplary” is used in the sense of “example” rather than “ideal.” The terms “comprises,” “comprising,” “includes,” “including,” or other variations thereof, are intended to cover a non-exclusive inclusion such that a composition, method, or process that comprises a list of elements or steps does not necessarily include only those elements or steps, but may include other elements or steps not expressly listed or inherent to such a composition, method, or process. The relative terms, such as “approximately” and “about,” are generally used to indicate a possible variation of ±10% of a stated or understood value unless indicated otherwise in the specification. In addition, the term “between” used in describing ranges of values is intended to include the minimum and maximum values described herein. The use of the term “or” in the claims and specification is used to mean “and/or” unless explicitly indicated to refer to alternatives only or the alternatives are mutually exclusive, although the disclosure supports a definition that refers to only alternatives and “and/or.” As used herein “another” may mean at least a second or more.

Embodiments of this disclosure may be drawn to a dehydrated multi-layer placental amnion or amnion/chorion product, e.g., sheet, that may be configured to be placed around injured tissue, for example, injured nerves, including injured peripheral nerves. The multi-layer amnion or amnion/chorion product, e.g., sheet, may be used as a tissue covering to serve as an anatomical barrier to help provide protection from the surrounding environment.

Examples of tissue with which multi-layer amnion or amnion/chorion products described herein may be used include nerve tissue, such as peripheral nerve tissue or central nervous system tissue. Other types of tissue suitable for the present disclosure include, but are not limited to epithelial tissue, connective tissue, muscular tissue, tendon tissue, ligament tissue, vascular tissue, intestinal tissue (including but not limited to forestomach), dermal tissue, and cardiac tissue.

It is noted that although much of this disclosure discusses the multi-layer product being formed of amnion or of amnion and chorion, this disclosure is not so limited. For example, the multi-layer product may be formed of layers of one or more different types of tissue, for example, including but not limited to the various types of tissues mentioned in the prior paragraph above. Further, the tissue may include mammalian tissue, including human tissue and tissue of other primates, rodent tissue, equine tissue, canine tissue, rabbit tissue, porcine tissue, ovine, or other ruminant tissue. In addition, the tissue may be non-mammalian tissue, selected from piscine, amphibian, or insect tissue. The tissue may be allogeneic or xenogeneic to a subject into which the graft is implanted. The tissue may be a synthetic tissue, such as, but not limited to, laboratory-grown or 3D-printed tissue. These tissue types may be used in place of or in addition to amnion or amnion and chorion tissue layers, such as those described herein. Products made of the alternative tissue types discussed in this paragraph and/or the preceding paragraph may include a similar number of layers as those described herein in regards to multi-layer amnion products.

The multi-layer amnion (MLA) product may be a dehydrated sheet composed of at least 4 layers, for example, from 4 to 10 layers, from 4 to 7 layers, at least 4 layers and less than 10 layers of placental sac amnion or amnion and chorion membranes, as will be discussed further below. The term multi-layer amnion or MLA refers to either a tissue product comprised of all amnion layers or a tissue product comprised of amnion and chorion layers. In both product types, the outermost layers (e.g., the top and bottom layers) are amnion layers. In some aspects, the multi-layer amnion product may be a dehydrated sheet composed of at least 5 layers, for example, from 5 to 10 layers, from to 7 layers, or from 5 to 6 layers of placental sac amnion or amnion and chorion membranes. The placental sac amnion and/or chorion membranes may be obtained from a human source, or, alternatively, a non-human source, or a mixture of human and non-human sources. In some embodiments, the layers of may be alternating human placental sac amnion and chorion membranes.

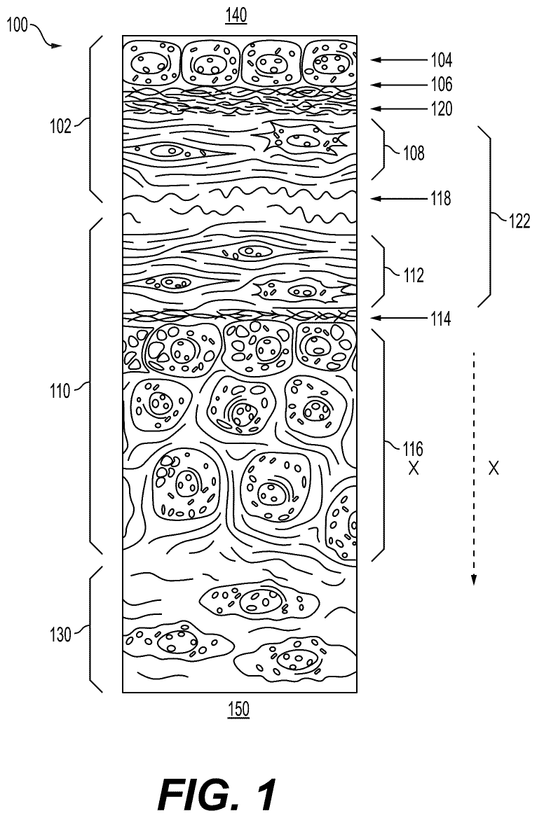

The amnion and chorion membrane layers of the present disclosure are component layers of the cross-section of human amniotic placental sac 100 shown in FIG. 1. An amnion membrane layer 102 as used herein refers to the parts shown in FIG. 1, including an epithelial layer 104, a basement membrane 106, and a fibroblast layer 108. A chorion membrane layer 110 as used herein includes a reticular layer 112 and, optionally, a basement membrane 114, but ideally omits most of, if not all, of the cells of a trophoblast layer 116. For example, during harvesting, the chorion membrane layer 110 may be separated from the cells of the trophoblast layer 116. As shown, a compact layer 120 may separate the basement membrane 106 of the amnion membrane layer 102 from the fibroblast layer 108. Additionally, a sponge layer 118 may be positioned between the amnion membrane layer 102 and the chorion membrane layer 110 (e.g., between the fibroblast layer 108 and the reticular layer 112). The fibroblast layer 108, the sponge layer 118, and the reticular layer 112 are mesenchymal cells 122. A first side of the placental sac 100, for example, the top side in FIG. 1 (e.g., an outer portion of the epithelial cells in epithelial layer 104 of amnion membrane layer 102) may be adjacent to an amniotic cavity 140. A second side of the placental sac 100, for example, the bottom side in FIG. 1 (e.g., an outer portion of a decidua layer 130) may be adjacent to a uterus 150.

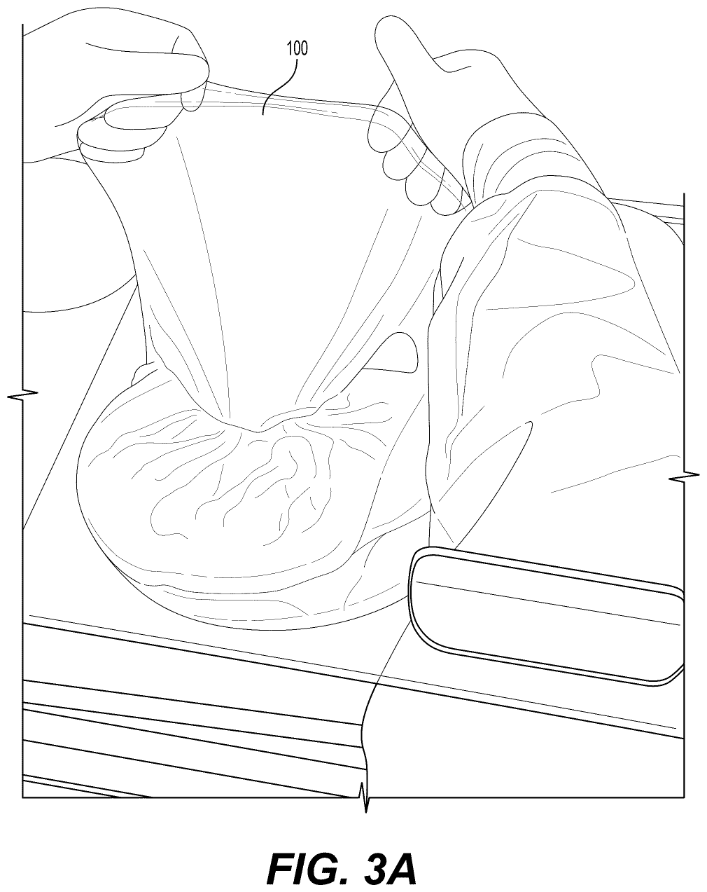

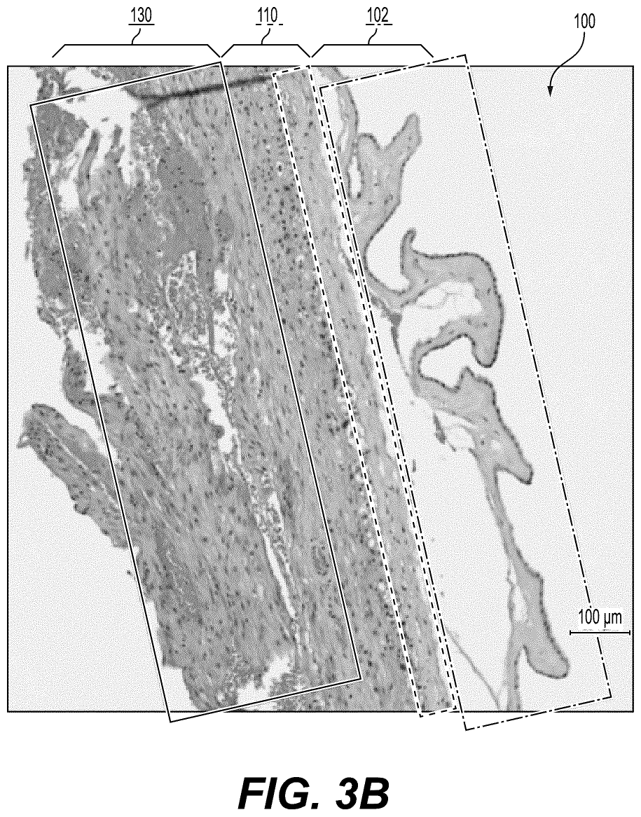

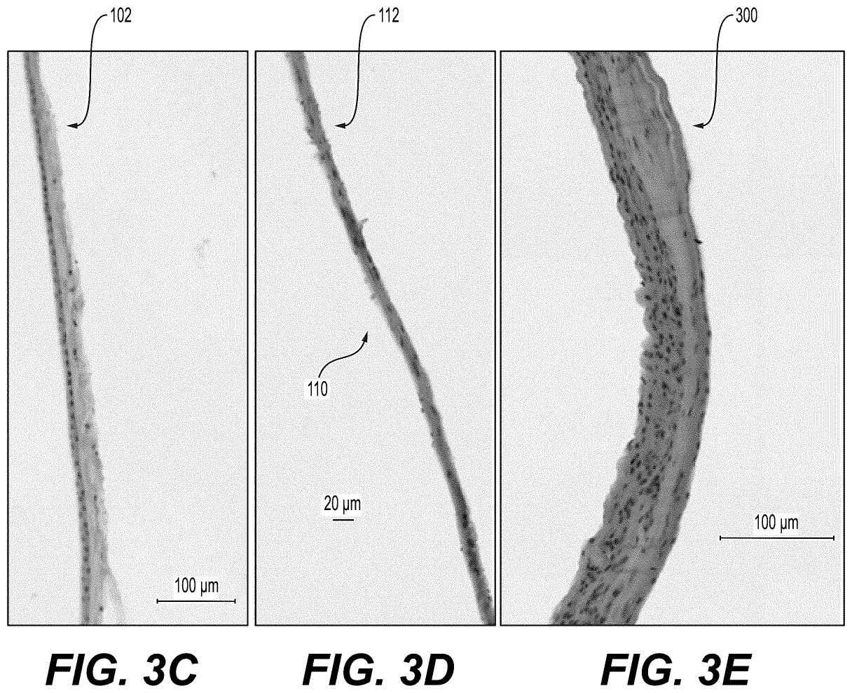

Amnion layers 102 may be harvested from the amniotic placental sac 100 (FIG. 3A) by physical separation at the sponge layer 118, for example. Chorion layers 110 may be harvested by physical separation from the decidua layer 130 of the amniotic sac at the trophoblast layer 116. Trophoblast cells that remain on the harvested chorion layer 110 may be substantially removed. To harvest the layers, the placenta may be oriented so that the amnion membrane layer is facing out. Manual debridement may then be performed to separate the amnion. If chorion layers are also to be harvested, they may also be manually separated from the placental tissue. A cell scraper or other suitable tool may also be used during debridement.

Removed amnion layers 102 and chorion layers 110 may be washed following separation from the placental sac 100. In some embodiments, the amnion membrane layer 102 or chorion membrane layer 110 may be cut before washing. For example, one or more templates may be used to identify portions of the layers that may be cut to suitable sizes for use, and the tissue layers may be cut according to the templates. The amnion or amnion and chorion layers may be cut with a scalpel blade, a die cutter, pneumatic press, or any other suitable instrument. In some embodiments, the amnion membrane layer 102 or chorion membrane layer 110 may be cut after washing. In other embodiments, the amnion membrane layer 102 or chorion membrane layer 110 may be cut before and after washing. In some embodiments, the amnion membrane layer 102 or chorion membrane layer 110 may be washed before being cut and after being cut.

After harvest, the amnion membrane layer 102 and chorion membrane layer 110 may be washed using one or more solutions to clean the amnion membrane layer 102 and chorion membrane layer 110. In one embodiment, two solutions may be used to clean one or more of the amnion membrane layer 102 or chorion membrane layer 110. The first wash solution may contain about 0.5% polyoxyethylene sorbitol ester such as Tween 20 (v/v), about 0.05% polyhexamethylene biguanide (PHMB) (w/v) or alternately about 0.5% chlorhexidine (w/v), about 0.9% NaCL (w/v), about 10 mM Bis-Tris Propane, and a solution of about pH 6.7+/−0.1. In another example, the first solution may contain about 0.5% polyoxyethylene sorbitol ester such as Tween 20 (v/v), and about 10.0% NaCl (w/v), having a pH of about 4.5-7.0, (e.g., a pH of 5.6). The second wash solution may contain about 0.144 g/L KH₂PO₄, about 9 g/L NaCl, and about 0.795 g/L Na₂HPO₄. In another example, the second solution may contain about 0.9% NaCl (w/v), having a pH of about 4.5-7.0, (e.g., a pH of 5.6).

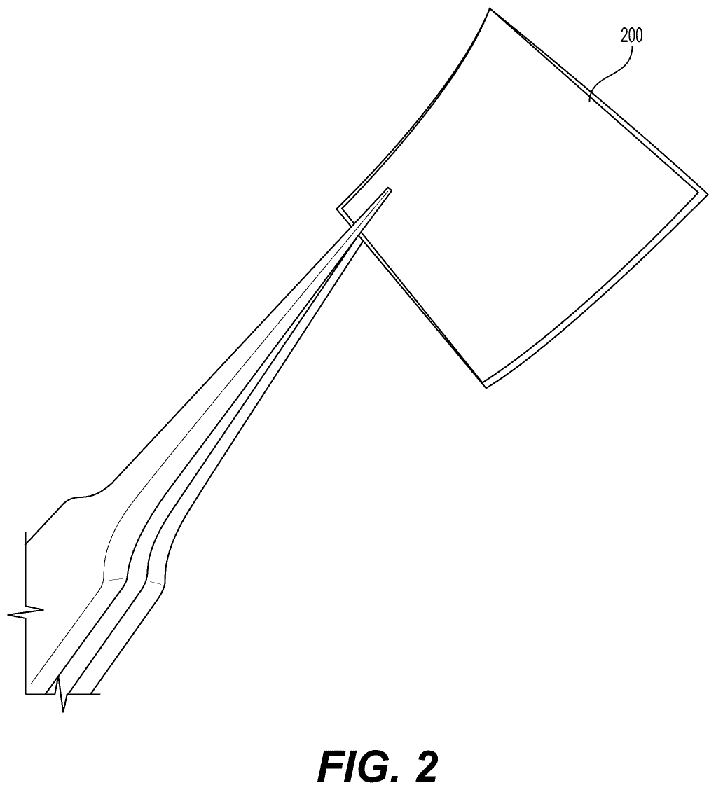

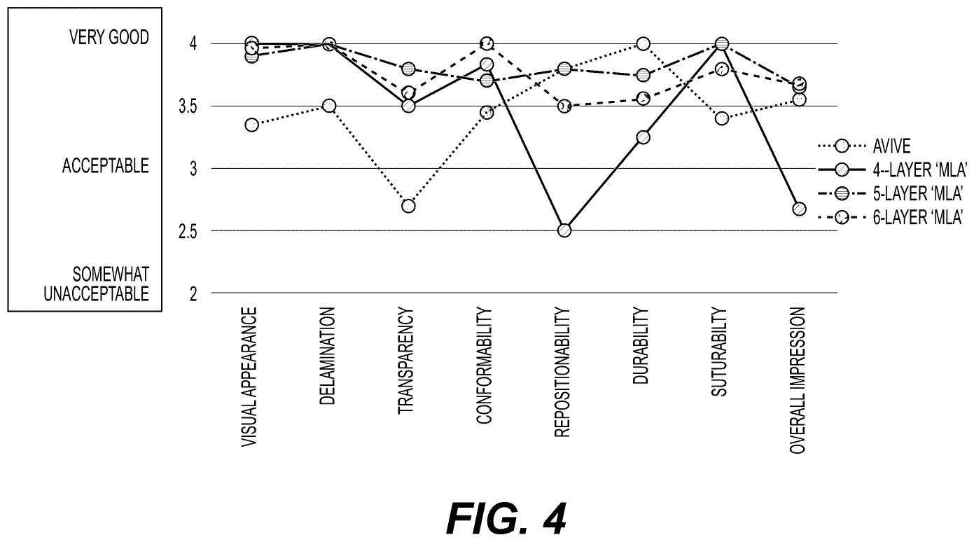

After washing, the amnion or amnion and chorion membrane layers may be cut, e.g., if they have not been cut before washing, and overlaid upon one another to create a multi-layer amnion product 200, such as the one shown in FIG. 2. In some aspects, the multi-layer amnion product may include a minimum of 4 total layers of either amnion or amnion and chorion. In some embodiments, the amnion or amnion and chorion membrane layers may be overlaid upon one another to create 4 to 10 layers, or 4 to 7 layers, for example, 4 layers, 5 layers, 6 layers, or 7 layers. The number of layers of the multi-layer amnion product may affect the handling properties of the multi-layer amnion product, as described further below in reference to FIG. 4. One or more support structures (e.g., a layering tool) may be used to help overlay and/or position the amnion or amnion and chorion membrane layers.

The multi-layer amnion product may contain an amnion membrane layer 102 on one exposed side that is oriented such that the epithelial layer 104 is facing out. The opposite exposed side of the multi-layer amnion product may contain another amnion membrane layer 102 that may also be oriented with the epithelial layer 104 facing out. Accordingly, the orientation of the outermost layers of the MLA product may be such that an amnion layer 102 is the outermost layer on both sides of the multi-layer amnion product. The outermost amnion layer 102 on both sides of the multi-layer amnion product, e.g., sheet, may be oriented such that the epithelial layer 104 is facing outward. Thus, for a sheet having a top surface and a bottom surface, the top surface may include epithelial cells, and the bottom surface may include epithelial cells. In these aspects, the epithelial layer 104 may be smoother and/or more slippery than the inner facing layers of the amnion membrane layer 102 and of the chorion membrane layer 110. Having both the top surface and the bottom surface including epithelial cells from the epithelial layer 104 may allow the multi-layer amnion product to not have a “sidedness.” For example, the multi-layer amnion may be applied with either the top or bottom surface facing towards or away from the tissue to which it is applied during use.

An amnion membrane layer has two sides. One side includes epithelial cells, and the other side includes the fibroblast layer. When amnion membrane layers are layered upon one another to form the multi-layer amnion product, these adjacent amnion layers may be oriented so that the epithelial portion of a first amnion layer is adjacent the fibroblast portion of a second amnion layer, or so that the fibroblast portion of a first amnion layer is adjacent the fibroblast portion of a second amnion layer. The adjacent first and second amnion layers may not be layered in the epithelial-epithelial orientation in order to avoid delamination.

In some embodiments, the organization of layers within the multi-layer amnion product may be such that amnion layers and chorion layers are repeating, i.e., like layers are organized to be adjacent. For example, combinations of layers for the 5-, 6-, or 7-layer MLA product may be organized as follows:

5-layer:
AACCA, AACAA, AAACA, ACACA, AAAAA, or ACCCA
6-layer:
AACCAA, AAACCA, AAAACA, AACAAA, ACCCAA, ACCCCA,
or AAAAAA
7-layer:
AAACAAA, AAAACAA, AAAAACA, AACCAAA, AACCCAA,
AAACCCA, AACCCCA, ACCCCCA, or AAAAAAA

In the above examples, each “A” represents an amnion layer (e.g., amnion membrane layer 102), and each “C” represents a chorion layer (e.g., chorion membrane layer 110). Each amnion layer may include an epithelial layer (e.g., epithelial layer 104), a basement membrane (e.g., basement membrane 106), and a fibroblast layer (e.g., fibroblast layer 108), as indicated above. Each chorion layer may include a reticular layer (e.g., reticular layer 112). In some embodiments, each chorion layer may include a basement layer in addition to the reticular layer. In some embodiments, one or more chorion layers, but not all chorion layers, include a basement layer (e.g., basement membrane 114) in addition to the reticular layer. In any of these embodiments, the one or more chorion layers may have had trophoblast cells (e.g., trophoblast layer 116) substantially removed.

In some embodiments, the overlaid layers of tissue, including but not limited to overlaid layers of amnion, or overlaid layers of amnion and chorion, may then be dried to form a dehydrated sheet. For example, the overlaid layers may be dried using a vacuum (e.g., at a pressure less than atmospheric pressure) at a temperature ranging from about 18 degrees Celsius to about 35 degrees Celsius, e.g., from about 22 degrees Celsius to about 35 degrees Celsius, to dry the multi-layer tissue, or for example, the multi-layer amnion product, although any suitable drying method may be used. Exemplary vacuum pressure used for drying may be, e.g., about 50 mbar to about 350 mbar.

The subsequent dehydrated tissue sheet composed from amnion or amnion and chorion membrane layers may then be sterilized using a suitable method. In one embodiment, e-beam radiation may be used to achieve sterility assurance of about 10⁻⁶.

A method of forming a multi-layer amnion product may include separating amnion and chorion layers from the placenta and amniotic sac, washing the amnion and chorion layers, layering them upon one another in alternating or repeating order, and dehydrating the amnion and chorion layers after being stacked upon one another. In embodiments in which only amnion layers are used, the method of forming a multi-layer amnion product may include separating amnion layers from the placenta and amniotic sac, washing the amnion layers, layering them upon one another, and dehydrating the amnion layers after being stacked upon one another. For example, a method of forming a multi-layer amnion product may include one or more of harvesting the amniotic sac and placenta, and separating the chorion layer from the decidua at the trophoblast layer. The amnion layer may include all, at least some, or none of the sponge layer. If chorion layers are used, trophoblast cells on the chorion layer may be substantially removed. For example, the chorion layer to be used in the multi-layer amnion product may include no trophoblast cells. In other aspects, the chorion layer to be used in the multi-layer amnion product may include some trophoblast cells. Furthermore, in various aspects, the chorion layer may include all, at least some, or none of the sponge layer.

The method may also include washing the amnion or amnion and chorion membrane layers using one or more cleaning solutions, for example, two cleaning solutions, examples of which are described above. The cut and washed layers may be overlaid upon one another to form a multi-layer amnion product, e.g., sheet. The multi-layer amnion product may include at least 4 layers or at least 5 layers, for example, 4 to layers, or 4 to 7 layers, 5 to 10 layers, 5 to 7 layers, or 5 to 6 layers, e.g., 4 layers, 5 layers, 6 layers, or 7 layers. The layers may be oriented so that the amnion layers are the outermost layers on both sides of the multi-layer sheet. The outermost amnion layers on both sides (e.g., top and bottom layers) may be oriented with the epithelial layer facing outward. To the extent to which there are adjacent amnion layers within the multi-layer amnion product, the adjacent amnion layers may be oriented so that the epithelial layer of a first adjacent amnion layer is facing the fibroblast layer of the second adjacent amnion layer, or so that the fibroblast layer of the first adjacent amnion layer is facing the fibroblast layer of the second adjacent amnion layer. The multi-layer amnion product may then be dried to form a dehydrated sheet. In some embodiments, drying the multi-layer amnion product may include vacuum drying at a vacuum pressure of about 50 mbar to about 350 mbar at a temperature of about 22 degrees Celsius to about 35 degrees Celsius. The multi-layer amnion product may be sterilized, e.g., using e-beam radiation, or other suitable sterilization methods.

FIG. 2 shows a sample 7-layer multi-layer amnion prototype 200. FIG. 3A shows a human placental amniotic sac, for example, human amniotic placental sac 100. FIG. 3B shows a hematoxylin and eosin stain histological image of harvested amniotic placental sac 100 with all layers present, for example, including at least amnion layer 102, chorion layer 110, and decidua layer 130. FIG. 3C shows a hematoxylin and eosin stain histological image of harvested isolated amnion membrane layer 102. FIG. 3D shows a hematoxylin and eosin stain histological image of harvested isolated reticular layer 112 of chorion layer 110. FIG. 3E shows a hematoxylin and eosin stain histological image of a 5-layer multi-layer amnion product 300. As discussed above, a multi-layer amnion product may include a plurality of only amnion layers 102 or in combination with one or more chorion layers 110 (e.g., reticular layer 112 of chorion layer 110).

FIG. 4 is a graph comparing various properties of multi-layer amnion products with different numbers of layers. As shown, the tested multi-layer amnion products include a four-layer MLA product, a five-layer MLA product, and a six-layer MLA product. The four-layer MLA product, the five-layer MLA product, and the six-layer MLA product may be formed via the steps and processes discussed herein. The four-layer MLA product was formed of amnion and chorion layers in the orientation AACA, wherein “A” denotes amnion and C denotes chorion. The five-layer MLA product was formed of amnion and chorion layers in the orientations AACAA and AACCA, wherein “A” denotes amnion and C denotes chorion. The six-layer MLA product was formed of amnion and chorion layers in the orientations AAACCA and ACACAA, wherein “A” denotes amnion and C denotes chorion. A dehydrated umbilical cord membrane product was also tested for comparison. The dehydrated umbilical cord membrane product was approximately 3 cm×6 cm, and the four-, five-, and six-layer MLA products were approximately 4 cm×8 cm.

As shown, each of the multi-layer amnion products was evaluated or assessed for visual appearance, delamination, transparency, conformability, repositionability, durability, suturability, and overall impression. Each of the multi-layer amnion products was evaluated on a scale, for example, between very good (e.g., 4), acceptable (e.g., 3), somewhat unacceptable (e.g., 2), and unacceptable (e.g., 1, not shown in FIG. 4).

The evaluation for visual appearance was based on the users' visual inspections of the tissue layers of the multi-layer amnion products. For example, the tissue layers should be a pale yellow or white color, and it was determined whether any discoloration was present. Visual inspection was made as to whether any red blood streaks were present, whether the color of the tissue was consistent, and whether there were any holes or tears in the tissue.

The evaluation for delamination was based on whether the tissue layers of the multi-layer amnion products delaminated (e.g., separated from an adjacent tissue layer) when handled. In these aspects, less delamination may help to ensure that the multi-layer amnion product retains its shape and inter-layer connections when handled. Delamination may negatively impact the user's ability to handle and/or apply the multi-layer amnion products. A score of 4 corresponds to no delamination; a score of 3 corresponds to some delamination visible, but tissue largely unchanged (e.g., corners or edges of the tissue product coming apart, but overall the tissue product still being usable); a score of 2 corresponds to significant delamination; and a score of 1 corresponds to full delamination (tissue product unusable and layers fully separated).

The evaluation of transparency was based on how transparent the tissue (e.g., the tissue that forms the multi-layer amnion product) was, for example, after hydration. Transparency may help the user visualize and/or position tissue (e.g., nerve tissue) beneath and/or surrounded by the multi-layer amnion product. Accordingly, transparency may make the multi-layer amnion product easier to use. A score of 4 corresponds to the tissue being transparent; a score of 3 corresponds to the tissue being somewhat transparent; a score of 2 corresponds to the tissue being somewhat opaque; and a score of 1 corresponds to the tissue being opaque. In use, transparency may allow for a user to see the underlying structures to which the MLA product is being applied or wrapped around. This may make the MLA product easier to use, for example, in the case of nerve coaptation, since the user may be able to visualize where nerve ends are positioned within the MLA product and relative to one another.

The evaluation for conformability was based on how satisfactory the conformability or drapability (e.g., draping across a midline) of the tissue was. A greater amount of conformability or drapability may help the multi-layer amnion product to surround or conform to the shape or size of the tissue (e.g., nerve tissue). For example, a score of 4 corresponds to satisfactory, drapes completely; a score of 3 corresponds to somewhat satisfactory draping; a score of 2 corresponds to somewhat unsatisfactory draping; and a score of 1 corresponds to unsatisfactory draping; like a stiff sheet.

The evaluation of repositionability was based on the ability of the tissue (e.g., the tissue that forms the multi-layer amnion product) to be straightened and/or repositioned after folding. For example, a score of 4 corresponds to no folding or clumping occurring, a score of 3 corresponds to some folding or clumping occurring but the folding or clumping is relatively easy to manipulate; a score of 2 corresponds to the tissue partially folding or clumping together; and a score of 1 corresponds to the tissue completely folding in on itself. In these aspects, a greater amount of repositionability may help the multi-layer amnion product to be straightened and/or repositioned, for example, to be more suitably positioned on tissue.

The evaluation of durability was based on the rated durability of the tissue (e.g., the tissue that forms the multi-layer amnion product). For example, the durability assessment of the tissue includes tugging on the tissue (e.g., tugging on a suture that has been passed through the tissue) and evaluating the ability of the tissue to withstand tearing of the suture through the tissue. For example, a score of 4 corresponds to the tissue being very durable; a score of 3 corresponds to the tissue being durable; a score of 2 corresponds to the tissue being somewhat durable (e.g., with some tearing seen); and a score of 1 corresponds to the tissue being not durable (e.g., the tissue falling apart). In these aspects, a greater amount of durability may help the multi-layer amnion product to be positioned, sutured, pulled, and/or otherwise manipulated multiple times and/or in multiple directions before, during, or after implantation into a patient.

The evaluation of suturability was based on the rated suturability of the tissue (e.g., the tissue that forms the multi-layer amnion product). For example, the rated suturability of the tissue includes whether it was possible to pass a suture through the tissue, and/or whether the tissue tears during suturing. A high score corresponded to both the ability to push a suture through the tissue product and the resistance to tearing or falling apart once the suture has passed through. For example, a score of 4 corresponds to satisfactory suturing (e.g., the suture passed through easily and then stayed in place and did not tear through the tissue product); a score of 3 corresponds to somewhat satisfactory suturing (slightly harder to pass the suture through and/or tore slightly); a score of 2 corresponds to somewhat unsatisfactory suturing (harder to pass the suture through and/or tore more); and a score of 1 corresponds to unsatisfactory suturing (e.g., suture didn't pass through and/or went right through and tore). In these aspects, a greater amount of suturability may help the multi-layer amnion product to be sutured to the tissue (e.g., nerve tissue) and/or to itself, for example, to surround tissue.

The evaluation of overall impression was based on the rated overall handling of the multi-layer amnion product. For example, the rated overall impression includes an evaluation of the usability of the multi-layer amnion product. For example, a score of 4 corresponds to the tissue product being very usable; a score of 3 corresponds to the tissue bring usable; a score of 2 corresponds to the tissue being somewhat usable; and a score of 1 corresponds to the tissue being unusable. In these aspects, a greater amount of usability may help the multi-layer amnion product to be used to treat tissue (e.g., nerve tissue).

As shown in FIG. 4, the five-layer MLA product exhibited consistently high scores across the various categories, for example, with scores above at least 3.5 in each category. The scores were based on a number of evaluations by users (e.g., surgeons) of each type of multi-layer amnion product. Additionally, users indicated that the four-, five-, and six-layer MLA products felt thinner and/or easier to manipulate than the dehydrated umbilical cord membrane product. Exemplary MLA products may have a thickness of about 30 microns to about 120 microns, e.g., about 90 microns to about 100 microns, or about 60 microns to about 80 microns. Furthermore, users indicated that the dehydrated umbilical cord membrane product is not used with sutures, but is instead secured using microclips, fibrin glue, or by wrapping around itself in order to secure the dehydrated umbilical cord membrane product, as the thickness and other aspects may be less consistent with the dehydrated umbilical cord membrane product. By contrast, the four-, five-, and six-layer MLA products exhibited very good or very satisfactory suturing scores.

In other aspects, the four-, five-, and six-layer MLA products were more consistent in appearance and/or transparency than the dehydrated umbilical cord membrane product. Moreover, there were limited or no delamination concerns with the four-, five-, and six-layer MLA products. In these aspects, delamination may result in the separation of adjacent layers in the MLA products, which may negatively affect the handlability, durability, and/or one or more other properties of the MLA product. The four-layer MLA product was rated as being somewhat unacceptable in terms of repositionability and overall impression. The five- and six-layer MLA products both received highly acceptable ratings across all categories or attributes. Based on the assessments, the five-layer MLA product received the best overall feedback, for example, balancing the trade-off between handling qualities and the amount (i.e., number of layers) of tissue being implanted/resorbed.

Overall, it was found that if too many layers were used in the multi-layer amnion product, the resulting product would not drape or conform properly. Alternatively, if too few layers were used, then handleability was decreased, as the resulting product may tear or rip and may lack durability, repositionability, and/or suturability. Such characteristics may not be as important for external wound-care products, but embodiments of the disclosure may be suitable for use with internal tissues, such as nerves. Forming products with more layers thus makes it easier for a surgeon to handle, position, and/or secure multi-layer amnion products of the disclosure relative to delicate tissue structures, such as nerves, while still maintaining sufficient drape to, e.g., wrap around a nerve.

In these aspects, the four-, five-, and six-layer MLA products have the trophoblast layer 116 of the chorion membrane layer 110 substantially removed. As such, the four-, five-, and six-layer MLA products are composed of birth tissue membranes and minimal presence of disorganized tissues. Other publicly available amnion products largely are not composed of birth tissue membranes with minimal or no disorganized tissues. MLA products of the present disclosure may be more robust, have more of a consistent thickness and/or may be thicker than available products, and may retain its shape better than available products, which may crumple and have little or no durability.

A layering tool may be used to help support and/or hold various layers of tissue, for example, to form the MLA products discussed herein. For example, four or more layers of amnion and/or chorion may be sequentially positioned one on top of the other.

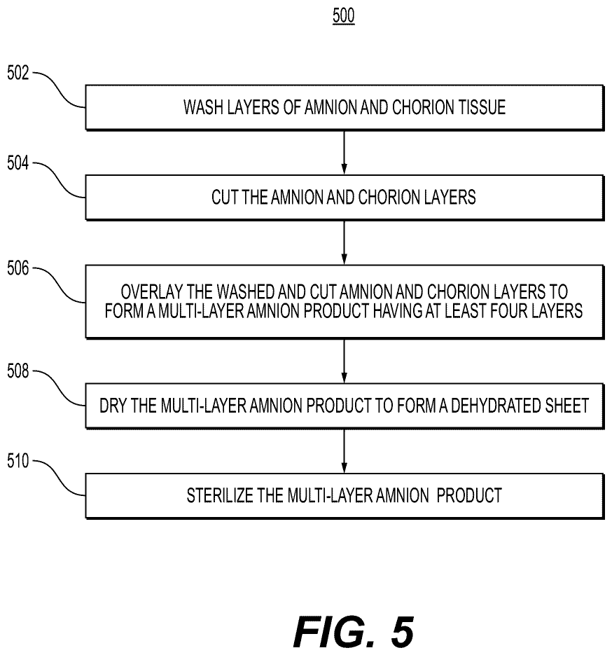

FIG. 5 is a flow chart of an exemplary method 500 of forming or making a multi-layer amnion product. Although not shown, method 500 may include one or more initial and/or optional steps. For example, method 500 may include initial steps of collecting and debriding placental tissue. The initial steps may include collecting amnion or amnion and chorion from the placental membrane. The initial steps may include debriding the chorion tissue, if included, to retain the reticular layer and basement membrane, while also substantially removing the trophoblast layer. The initial steps may include sizing the amnion and chorion from the placental membrane. For example, layers of amnion or amnion and chorion may be positioned over a layering tool.

Method 500 includes a step 502 that includes washing or otherwise performing solution processing procedures on layers of amnion or amnion and chorion tissue. As discussed above, the washing may include using one or more wash solutions. For example, the first wash solution may contain about 0.5% polyoxyethylene sorbitol ester such as Tween 20 (v/v), about 0.05% polyhexamethylene biguanide (PHMB) (w/v) or alternately about 0.5% chlorhexidine (w/v), about 0.9% NaCL (w/v), about 10 mM Bis-Tris Propane, and a solution of about pH 6.7+/−0.1. In another example, the first solution may contain about 0.5% polyoxyethylene sorbitol ester such as Tween 20 (v/v), and about 10.0% NaCl (w/v), having a pH of about 4.5-7.0, (e.g., a pH of 5.6). The second wash solution may contain about 0.144 g/L KH₂PO₄, about 9 g/L NaCl, and about 0.795 g/L Na₂HPO₄. In another example, the second solution may contain about 0.9% NaCl (w/v), having a pH of about 4.5-7.0, (e.g., a pH of 5.6).

Step 502 may include thawing frozen amnion or amnion and chorion segments, e.g., in a solution. Thawing may be done for at least four hours in ambient temperature (e.g., 15° C.-30° C.). For example, thawing may be done for between approximately 4 hours and approximately 18 hours, for example, approximately 4 hours and approximately 12 hours, approximately 14 hours and approximately 18 hours, or approximately 6 hours to approximately 8 hours. In some aspects, the frozen amnion or amnion and chorion segments may be thawed at refrigeration temperatures (e.g., 2° C.-10° C.) for about 48 hours to about 72 hours.

Additionally, step 502 may include washing the amnion or amnion and chorion layers one or more times, for example, in a saline solution. Step 502 may include incubating and/or agitating the amnion or amnion and chorion layers in one or more solutions. For example, in some aspects, step 502 may include incubating the amnion or amnion and chorion layers in a first solution at ambient temperature (e.g., between approximately 15 degrees Celsius and approximately 30 degrees Celsius) for a period of 15 minutes to approximately 2 hours, for example, 20 minutes to approximately 2 hours, 30 minutes to approximately 1.5 hours, approximately one hour to approximately 1.5 hours. The incubation may also include agitation, for example, at approximately 80 rpm to approximately 100 rpm, for example, approximately 90 rpm to approximately 95 rpm. Step 502 may also include an optional hold step in the incubator, for example, for approximately 30 minutes to approximately 60 minutes, for example, for approximately 40 minutes.

Moreover, in some aspects, step 502 may include one or more additional incubating procedures. For example, step 502 may also include incubating the amnion or amnion and chorion layers in a second solution at ambient temperature (e.g., between approximately 15 degrees Celsius and approximately 30 degrees Celsius) for a period of approximately 5 minutes to approximately 1 hour, for example, approximately 15 minutes to approximately 40 minutes. The incubation in the second solution may also include agitation, for example, at approximately 80 rpm to approximately 100 rpm, for example, approximately 90 rpm to approximately 95 rpm. Step 502 may also include an optional hold step in the incubator with the second solution, for example, for approximately 30 minutes to approximately 60 minutes, for example, for approximately 40 minutes. One or more of these incubating procedures may be repeated, for example, the second incubating procedure may be repeated, for example, repeated three or more additional times.

Method 500 includes a step 504 that includes cutting the amnion or amnion and chorion layers. The amnion or amnion and chorion layers may be cut to a desired size and/or shape. In some aspects, one or more templates may be arranged onto the tissue, and then the tissue may be cut according to the template arrangement. Example template sizes may be, e.g., about 8×8 cm, about 10×10 cm, or about 12×12 cm. The amnion or amnion and chorion layers may be cut with a scalpel blade, a die cutter, pneumatic press, or any other suitable instrument. In some aspects, the cutting described herein may be performed prior to the washing step 502. Alternatively, the multi-layer amnion product may be formed (e.g., via various steps of the methods discussed herein), and then the multi-layer amnion product may be cut to the desired size and/or shape.

Method 500 also includes a step 506 that includes overlaying the washed and cut amnion or amnion and chorion layers to form a multi-layer amnion product having at least four or at least five layers, e.g., four to ten layers, four to seven layers or five to seven layers. A layering tool may be used to help support the amnion or amnion and chorion layers to form the multi-layer amnion product. In some aspects, a layer of biocompatible, non-placental material may be positioned between the bottom plate of the layering tool and the first amnion layer. The first amnion layer may be oriented so that the epithelial layer faces down, opposite of where further layers of tissue will be stacked.

If two amnion layers are layered one on top of another, the adjacent amnion layers may be oriented so that the epithelial portion of one adjacent amnion layer is facing the fibroblast portion of the other adjacent amnion layer, or oriented so that the fibroblast portion of one adjacent amnion layer is facing the fibroblast portion of the other adjacent amnion layer. Adjacent amnion layers may not be layered so that the epithelial portions face each other in order to avoid delamination. It is noted that the intermediate layers of chorion do not need to be oriented in a particular orientation or arrangement. Then, a final amnion layer may be positioned over the other layers of amnion or amnion and chorion. The final amnion layer may be oriented so that the epithelial layer faces up, away from the preceding layers of tissue.

In some aspects, a top plate of a layering tool may then be positioned on top of the layers of amnion or amnion and chorion, for example, to help retain and/or compress the stacked layers. In some aspects, a layer of biocompatible, non-placental material may be positioned between the top plate and the multi-layer amnion product. In some aspects, at least four or at least five layers of amnion or amnion and chorion may be overlaid, with epithelial cells of the outermost amnion layers facing outward on both sides of the overlaid layers. In some embodiments, each layer may be positioned on a biocompatible sheet of material, and the layer and the biocompatible sheet of material may be positioned on a layering tool. In some aspects, the biocompatible sheet of material may be removed after each layer is positioned on the layering tool.

Method 500 includes a step 508 that includes drying the stacked layers of amnion or amnion and chorion to form a dehydrated sheet of multi-layer amnion product. The drying may be performed via vacuum-drying. In these aspects, the layers of amnion or amnion and chorion may be positioned within a drying or vacuum bag. As an example, suitable drying or vacuum bags and methods of drying are described in U.S. Provisional Application No. 63/477,566, filed Dec. 29, 2022, herein incorporated by reference in its entirety.

In one or more aspects, the drying may be performed for between approximately 16 hours and approximately 24 hours (e.g., one day and/or overnight). The drying may be performed at a temperature between approximately 15 degrees Celsius and approximately 35 degrees Celsius. The drying may be performed with an air bleed flow rate between approximately 30 SLPH and approximately 340 SLPH, for example, approximately 50 SLPH. The drying may be performed at a vacuum pressure between approximately 10 inHg and approximately 28 inHg. In some aspects, the drying may be performed at a vacuum pressure of greater than 25 inHg. It is noted that the above drying steps and procedures are only exemplary, and that one or more different drying steps or procedures may be performed to dry the multi-layer amnion product. Moreover, although step 504 is shown as preceding steps 506 and 508, it is noted that step 504 may alternatively be performed after step 506 or after step 508. In some aspects, step 504 of cutting may again be performed after step 508, or after step 510, described below. For example, the first step 504 may be performed in order to cut amnion and/or chorion layers for layering relative to one another, and a second step 504 may be performed after the drying step 508 or after step 510 in order to cut the MLA product into specific product shapes and/or sizes.

Furthermore, method 500 includes a step 510 that includes sterilizing the multi-layer amnion product, for example, after drying the multi-layer amnion product. For example, after sufficient drying, the multi-layer amnion product may be sterilized, e.g., using e-beam radiation. Step 510 may also include placing (e.g., sealing) the multi-layer amnion product, for example, in at least one pouch. For example, the multi-layer amnion product may be placed in one or more foil pouches. In some aspects, the multi-layer amnion product may be placed in an inner foil pouch, which then may be placed into an outer foil pouch.

In some aspects, method 500 may include a second cutting step 504 after step 508 or 510. The dried and/or sterilized multi-layer amnion product may be cut, for example, into one or more pieces. For example, the multi-layer amnion product may be cut into interior portion(s) and sacrificial portions. For example, the multi-layer amnion product may be cut with a scalpel blade, cutting die, pneumatic die, or other suitable cutting device. Moreover, the multi-layer amnion product may be cut into different sizes or shapes, for example, with one or more cutting die. The sizes or shapes may correspond to suitable sizes or shapes for final tissue products. The cutting die may be different sizes and/or shapes, for example, approximately 4 cm×approximately 6 cm, approximately 3 cm×approximately 6 cm, approximately 3 cm×approximately 4 cm, approximately 2 cm×approximately 4 cm, approximately 2 cm×approximately 2 cm, etc. In some aspects, the various cutting procedures may be performed on a cutting surface.

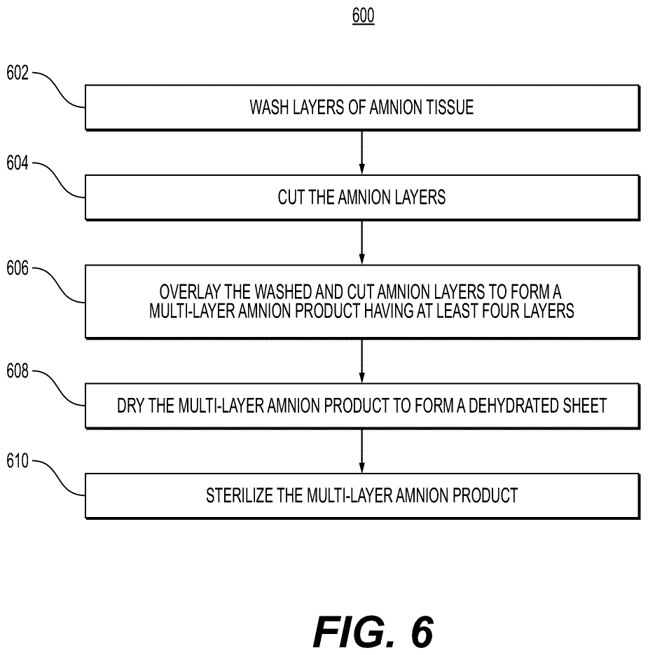

As shown in FIG. 6, a method 600 includes the various steps of method 500 may be performed with only layers of amnion, for example, to form or make a multi-layer amnion product without any layers of chorion. In these aspects, method 600 includes a step 602 that includes washing layers of amnion tissue. Method 600 also includes a step 604 that includes cutting the amnion layers. Method 600 includes a step 606 that includes overlaying the washed and cut amnion layers to form a multi-layer amnion product having at least four or at least five layers. Additionally, method 600 includes a step 608 that includes drying the multi-layer sheet to form a dehydrated sheet. Moreover, method 600 includes a step 610 that includes sterilizing the resulting multi-layer amnion product. One or more steps of method 600 may be performed in different orders and/or may be repeated (e.g., step 604), as described above in relation to method 500. Method 600 may be performed in a similar manner to method 500, except that method 600 may apply to amnion-only multi-layer products.

A multi-layer amnion product of the present disclosure (e.g., a multi-layer amnion dehydrated product) may act as a tissue covering for placement around injured tissue, for example, injured nerves, e.g., peripheral nerves. The multi-layer amnion product may consist of, for example, 4 to 10, 4 to 7, 5 to 10, or 5 to 7 isolated amnion or amnion and chorion membrane layers that have been harvested from, e.g., the human amniotic sac. As discussed above, the multi-layer amnion product may consist of five layers of isolated human amnion or amnion and chorion membrane layers that have been harvested from the human amniotic sac, or, alternatively, a non-human source. The amnion membrane layer may include an epithelial layer, a basement membrane, and a fibroblast layer. The chorion membrane layer (if included) may include a reticular layer and a basement membrane. In some aspects, these membranes may be layered into a multilayered graft in various arrangements as described above.

The multi-layer amnion product may be configured to be placed around injured peripheral nerves as a tissue covering to serve as an anatomical barrier to help provide protection from the surrounding environment. The multi-layer amnion product may be formed as a sheet and then may be rolled into a tube, cap, or other formation prior to placement around a tissue, or may be wrapped around the tissue during placement. The multi-layer amnion product may have any suitable size or shape, which may depend, at least in part, on the intended use, e.g., the size or type of tissue with which the product is intended to be used.

A method of repairing tissue using a multi-layer amnion product according to any one of the embodiments disclosed herein may include a step of orienting the MLA product at a coaptation or injury site adjacent two severed tissue ends or adjacent damaged tissue. The method may further include wrapping the MLA product around the injured tissue into a tube shape, if the MLA product is not already shaped as a tube. Once positioned around the injured tissue, the MLA product may be secured in place to the injured tissue. In some aspects, the damaged or injured tissue may be nerve tissue, e.g., peripheral nerve tissue.

The multi-layer amnion product may have superior handling properties around tissues, e.g., peripheral nerves, compared with other dehydrated amniotic sac based membranes. Exemplary superior handling properties may include one or more of the following: conformability around tissue such as a nerve, the ability to hold a suture, the ability to be moved freely as a sheet in both a dehydrated or rehydrated state, the ability to be repositioned after being placed on one or more tissues, etc. Such handling properties may not be as important when used for external purposes, e.g., on external wounds, but may be of particular interest when wrapping around or conforming to internal tissues like nerves. For example, properties like those tested in FIG. 4 may allow a surgeon to position, wrap, reposition, secure, visualize, etc., the multi-layer amnion product when positioning the product relative to a tissue within the body. Embodiments of the disclosure may be suitable for use with tissues within the body rather than only external wound care. Existing products may be unsuitable for these reasons with delicate internal tissues, such as nerves.

Further, multi-layer amnion products of the present disclosure may have resorption kinetics suitable for use with internal tissues within the body, e.g., for use with nerves. Embodiments of the disclosure may resorb after approximately four weeks, after approximately five weeks, after approximately six weeks, or after approximately seven weeks, e.g., in approximately four to eight weeks, or approximately six to eight weeks. While products used for application on wounds may be reapplied at regular intervals, products applied internally, e.g., to nerves, cannot be reapplied without surgical intervention. Accordingly, embodiments of the disclosure may have resorption times that allow them to stay in place for long enough for the tissue to heal.

The methods and multi-layer amnion products may also allow for one or more of the following: 1) debriding and separating layers of placental membrane without damaging the layers of placental membrane; 2) layering amnion or amnion and chorion membranes together in an effective way; 3) determining orientation of amnion layer (epithelial side vs. spongy side); 4) maximizing yield with overlapping layers of tissue (e.g., with four or more overlapping layers of amnion or layers of amnion and chorion).

Other embodiments of the disclosure will be apparent to those skilled in the art from consideration of the specification and practice of the invention disclosed herein. It is intended that the specification and examples be considered as exemplary only, with a true scope and spirit of the invention being indicated by the following claims.

What is claimed is:

1. A dehydrated multi-layer amnion product, comprising:
a plurality of layers oriented on top of one another, wherein the plurality of layers includes at least four layers and less than ten layers made up of at least one chorion membrane layer and at least two amnion layers, and wherein at least two of the at least two amnion layers form outermost layers of the plurality of layers,
wherein each amnion layer comprises an epithelial layer, a basement membrane, and a fibroblast layer, and
wherein each chorion layer comprises a reticular layer and a basement membrane.

2. The multi-layer amnion product of claim 1, wherein the plurality of layers comprises five, six, or seven layers.

3. The multi-layer amnion product of claim 1, comprising at least three amnion layers, wherein at least two of the at least three amnion layers are adjacent one another, and the adjacent amnion layers are oriented so that the epithelial layer of a first adjacent amnion layer is oriented facing the fibroblast layer of a second adjacent amnion layer.

4. The multi-layer amnion product of claim 1, comprising at least three amnion layers, wherein at least two of the at least three amnion layers are adjacent one another, and the adjacent amnion layers are oriented so that the fibroblast layer of a first adjacent amnion layer is oriented facing the fibroblast layer of a second adjacent amnion layer.

5. The multi-layer amnion product of claim 1, wherein each of the outermost amnion layers of the plurality of layers are oriented such that the epithelial layer is facing outward.

6. The multi-layer amnion product of claim 1, wherein the at least two amnion layers and the at least one chorion layer are obtained from a human source.

7. The multi-layer amnion product of claim 1, wherein the at least two amnion layers and the at least one chorion layer are obtained from a non-human source.

8. A method of making a multi-layer amnion product, comprising:
washing amnion and chorion layers, wherein the amnion and chorion layers are obtained from an amniotic sac and a placenta;
cutting the amnion and chorion layers;
overlaying the washed and cut amnion and chorion layers on top of one another to form a multi-layer sheet having at least four layers and less than ten layers, wherein the washed and cut amnion and chorion layers are oriented so that outermost layers of the multi-layer sheet are amnion layers;
drying the multi-layer sheet to form a dehydrated sheet; and
sterilizing the multi-layer sheet to form the multi-layer amnion product.

9. The method of claim 8, wherein washing the amnion and chorion layers includes using two cleaning solutions.

10. The method of claim 9, wherein a first cleaning solution of the two cleaning solutions comprises:
about 0.5% Tween 20 (v/v);
about 0.05% polyhexamethylene biguanide (w/v) or alternately about 0.5% (w/v) chlorhexidine;
about 0.9% NaCL (w/v);
about 10 mM Bis-Tris Propane; and
a solution of about pH 6.7+/−0.1.

11. The method of claim 9, wherein a first cleaning solution of the two cleaning solutions comprises:
about 0.5% Tween 20 (v/v); and
about 10.0% NaCl (w/v),
wherein the first cleaning solution has a pH of about 4.5-7.0.

12. The method of claim 9, wherein a second cleaning solution of the two cleaning solutions comprises:
about 0.144 g/L KH₂PO₄;
about 9 g/L NaCl; and
about 0.795 g/L Na₂HPO₄.

13. The method of claim 9, wherein a second cleaning solution of the two cleaning solutions comprises about 0.9% NaCl (w/v) and has a pH of about 4.5-7.0.

14. The method of claim 8, wherein at least two adjacent amnion layers are oriented adjacent one another such that a fibroblast layer of a first adjacent amnion layer is oriented facing a fibroblast layer of a second adjacent amnion layer, or the fibroblast layer of the first adjacent amnion layer is oriented facing an epithelial layer of a second adjacent amnion layer.

15. The method of claim 8, wherein the multi-layer sheet comprises five, six, or seven layers.

16. The method of claim 8, wherein the outermost amnion layers are oriented such that an epithelial layer of each of the outermost amnion layers is facing outward.

17. The method of claim 8, further comprising:
harvesting the amnion and chorion layers from the amniotic sac and placenta, wherein the harvesting includes:
separating the amnion layer at a sponge layer;
separating the chorion layer from a decidua of the amniotic sac at a trophoblast layer; and
substantially removing trophoblast cells remaining on the chorion layer.

18. The method of claim 8, wherein cutting the amnion and chorion layers occurs before washing the amnion and chorion layers.

19. The method of claim 8, wherein the multi-layer sheet comprises five layers, and wherein overlaying the washed and cut amnion and chorion layers on top of one another comprises orienting the amnion and chorion layers in one of the following arrangements:
AACCA;
ACACA;
AACAA;
AAACA;
or
ACCCA,
wherein each “A” represents an amnion layer and each “C” represents a chorion layer.

20. The method of claim 8, wherein the multi-layer sheet comprises six layers, and wherein overlaying the washed and cut amnion and chorion layers on top of one another comprises orienting the amnion and chorion layers in one of the following arrangements:
AACCAA;
AAACCA;
AAAACA;
AACAAA;
ACCCAA;
or
ACCCCA,
wherein each “A” represents an amnion layer and each “C” represents a chorion layer.

21. The method of claim 8, wherein the multi-layer sheet comprises seven layers, and wherein overlaying the washed and cut amnion and chorion layers on top of one another comprises orienting the amnion and chorion layers in one of the following arrangements:
AAACAAA;
AAAACAA;
AAAAACA;
AACCAAA;
AACCCAA;
AAACCCA;
AACCCCA;
or
ACCCCCA,
wherein each “A” represents an amnion layer and each “C” represents a chorion layer.

22. The method of claim 8, wherein sterilizing the multi-layer sheet comprises using e-beam radiation to achieve sterility assurance of about 10⁻⁶.

23. The method of claim 8, wherein drying the multi-layer sheet to form a dehydrated sheet comprises using a vacuum pressure of about 50 mbar to about 350 mbar at a temperature of about 22 degrees Celsius to about 35 degrees Celsius.

24. The method of claim 8, further comprising rolling the multi-layer sheet to form a tube.

25. A method of making a multi-layer amnion product, comprising:
washing a plurality of amnion layers, wherein the amnion layers are obtained from an amniotic sac;
cutting the amnion layers;
overlaying the washed and cut amnion layers on top of one another to form a multi-layer sheet having at least four layers and less than ten layers, wherein the washed and cut amnion layers are oriented so that outermost layers of the multi-layer sheet are amnion layers, and the outermost amnion layers are oriented such that an epithelial layer of each of the outermost amnion layers is facing outward;
drying the multi-layer sheet to form a dehydrated sheet; and
sterilizing the multi-layer sheet to form the multi-layer amnion product.

26. The method of claim 25, wherein the multi-layer sheet comprises five, six, or seven layers.

27. The method of claim 25, wherein adjacent amnion layers are oriented so that an epithelial layer of a first adjacent amnion layer is oriented facing a fibroblast layer of a second adjacent amnion layer.

28. The method of claim 25, wherein adjacent amnion layers are oriented so that a fibroblast layer of a first adjacent amnion layer is oriented facing a fibroblast layer of a second adjacent amnion layer.

29. A dehydrated multi-layer amnion product, comprising:
a plurality of layers oriented on top of one another, wherein the plurality of layers includes at least four amnion layers,
wherein each amnion layer comprises an epithelial layer, a basement membrane, and a fibroblast layer, and
wherein outermost layers of the plurality of layers are amnion layers, and wherein the outermost amnion layers are oriented such that the epithelial layer is facing outward.

30. The multi-layer amnion product of claim 29, wherein the plurality of layers consists of five amnion layers.

31. The multi-layer amnion product of claim 29, wherein the plurality of layers comprises four, five, six, or seven layers.

32. The multi-layer amnion product of claim 29, wherein adjacent amnion layers are oriented so that the epithelial layer of a first adjacent amnion layer is oriented facing the fibroblast layer of a second adjacent amnion layer.

33. The multi-layer amnion product of claim 29, wherein adjacent amnion layers are oriented so that the fibroblast layer of a first adjacent amnion layer is oriented facing the fibroblast layer of a second adjacent amnion layer.

34. The multi-layer amnion product of claim 29, wherein the amnion layers are obtained from a human source.

35. The multi-layer amnion product of claim 29, wherein the amnion layers are obtained from a non-human source.
